(19) United States
(12) Patent Application Publication
(10) Pub. No.: US 2025/0272554 A1
(43) Pub. Date: Aug. 28, 2025

(54) AUTOMATIC REFINEMENT OF ELECTROGRAM SELECTION

(71) Applicant: c/o Vektor Medical, Inc., San Diego, CA (US)

(72) Inventors: Christopher J. T. Villongco, San Diego, CA (US); Christian David Marton, Jersey City, NJ (US); Prateek Bhatnagar, San Diego, CA (US)

(21) Appl. No.: 18/878,007

(22) PCT Filed: Aug. 24, 2023
(86) PCT No.: PCT/US23/72866

§ 371 Date: Dec. 21, 2024

Related U.S. Application Data
(60) Provisional application No. 63/401,048, filed on Aug. 25, 2022.

(51) Int. Cl. G06N 3/08; G06N 3/0455; G06N 3/0464

(52) U.S. Cl. CPC G06N 3/08; G06N 3/0455; G06N 3/0464

(57) ABSTRACT

A system for identifying a base region of a cardiogram to inform treatment of a patient is provided. The system accesses a subject region of a subject cardiogram collected from a patient. The system then identifies a subject base region of the subject cardiogram based on mappings of derived regions of base cardiograms to base regions of base cardiograms. Each derived region is derived from the base region of a base cardiogram to which the derived region is mapped. The system then outputs an indication of the subject base region of the subject cardiogram to inform treatment of a patient.

### CROSS-REFERENCE TO RELATED APPLICATION

This application claims the benefit of U.S. Pro. App. No. 63/401,048 filed on Aug. 25, 2022 and entitled “AUTOMATIC REFINEMENT OF ELECTROGRAM SELECTION,” which is hereby incorporated by reference it its entirety.

### BACKGROUND

Many heart disorders can cause symptoms, morbidity (e.g., syncope or stroke), and mortality. Common heart disorders caused by arrhythmias include inappropriate sinus tachycardia (IST), ectopic atrial rhythm, junctional rhythm, ventricular escape rhythm, atrial fibrillation (AF), ventricular fibrillation (VF), focal atrial tachycardia (focal AT), atrial micro-reentry, ventricular tachycardia (VT), atrial flutter (AFL), premature ventricular complexes (PVCs), premature atrial complexes (PACs), atrioventricular nodal reentrant tachycardia (AVNRT), atrioventricular reentrant tachycardia (AVRT), permanent junctional reciprocating tachycardia (PJRT), and junctional tachycardia (JT). The sources of arrhythmias may include electrical rotors (e.g., ventricular fibrillation), recurring electrical focal sources (e.g., atrial tachycardia), anatomically based reentry (e.g., ventricular tachycardia), and so on. These sources are important drivers of sustained or clinically significant episodes. Arrhythmias can be treated with ablation using different technologies, including radiofrequency energy ablation, pulsed field ablation, cryoablation, ultrasound ablation, laser ablation, external radiation sources, directed gene therapy, and so on, by targeting the source of the heart disorder. Since the sources of heart disorders and the locations of the sources vary from patient to patient, even for common heart disorders, targeted therapies require the sources of the arrhythmias to be identified.

Unfortunately, common methods for reliably identifying the sources and the source locations of a heart disorder can be complex, cumbersome, and expensive. For example, one method uses an electrophysiology catheter having a multi-electrode basket catheter that is inserted into the heart (e.g., left ventricle) intravascularly to collect from within the heart measurements of the electrical activity of the heart, such as during an induced episode of VF. The measurements can then be analyzed to help identify a possible source location. Presently, electrophysiology catheters are expensive (and generally limited to a single use) and may lead to serious complications, including cardiac perforation and tamponade. Another method uses an exterior body surface vest with electrodes to collect measurements from the patient's body surface, which can be analyzed to help identify an arrhythmia source location. Such body surface vests are expensive, are complex and difficult to manufacture, and may interfere with the placement of defibrillator pads needed after inducing VF to collect measurements during the VF. In addition, the vest analysis requires a computed tomography (CT) scan and is unable to sense the interventricular and interatrial septa where approximately 20% of arrhythmia sources may occur.

Recently, technologies have been developed to aid in the identification of the source location of an arrhythmia. One technology runs simulations of the electrical activity of a heart that represent multiple cardiac cycles over time. Each simulation may simulate, for example, 10 seconds of cardiac activity or may run until a termination criterion has been satisfied (e.g., rotor stabilization). Each simulation assumes an arrhythmia originating from a source location based on a source configuration of the heart. A source configuration may include various cardiac characteristics such as arrhythmia type, arrhythmia source location, geometry and orientation of a heart, scar locations, prior ablation locations, action potential characteristics, conduction velocity, pacing lead locations, heart disease, and so on. This technology may run millions of simulations. each of which has a different source configuration.

For each simulation, this technology generates a simulated cardiogram (i.e., a waveform), such as an electrocardiogram (ECG) or vectorcardiogram (VCG), from the simulated electrical activity. This technology then generates mappings of the simulated cardiograms (e.g., simulated cycles or other simulated regions of the cardiograms) to the source locations used in the simulation from which the simulated cardiogram was generated. Aspects of this technology (e.g., a source location mapping system) are described in U.S. Pat. No. 10,860,754 entitled “Calibration of Simulated Cardiograms” and granted on Dec. 8, 2020, which is hereby incorporated by reference.

To identify a source location for a patient, a patient cardiogram is collected during an episode of an arrhythmia. This technology may display the patient cardiogram to an electrophysiologist (EP) and request that the EP select a region of the cardiogram covering an extent (e.g., start time to end time) of the patient cardiogram to be used by a source location mapping system to identify the source location. The selected region may be a T-Q interval relating to an atrial fibrillation, a QRS complex relating to a ventricular tachycardia, a region relating to a ventricular fibrillation induced by pacing, and so on. This technology may identify a simulated region of the mappings based on similarity to the selected region. The source location to which the identified simulated region is mapped is then employed to inform treatment of a patient.

Because millions of simulations may be run, it is possible that simulated regions of simulated cardiograms are similar to simulated regions of other simulated cardiograms even though the source locations of their simulations are somewhat different. The accuracy of the identification of a source location depends in part on how well the extent of a patient region corresponds to the extents of the simulated regions. For various reasons, different EPs may select different extents when provided the same patient cardiogram. As a result, the source locations that are identified may be somewhat different or even very different.

### BRIEF DESCRIPTION OF THE DRAWINGS

This application contains at least one drawing executed in color. Copies of this application with color drawing(s) will be provided by the Office upon request and payment of the necessary fees.

FIG. 1 is a flow diagram that illustrates the processing of an identify data component of the ARES system in some embodiments.

FIG. 2 is a block diagram that illustrates the components of the ARES system in some embodiments.

FIG. 3 illustrates some data structures and machine learning architectures of the ARES system in some embodiments.

FIG. 4 is a flow diagram that illustrates the processing of the generate mappings component of the ARES system.

FIG. 5 is a flow diagram that illustrates the processing of an identify source locations components of the ARES system in some embodiments.

FIG. 6 is a flow diagram that illustrates the processing of a display source locations component of the ARES system in some embodiments.

FIG. 7 is a flow diagram that illustrates the processing of a display lead source locations component of the ARES system in some embodiments.

FIG. 8 illustrates examples of automatically detected arrhythmic and normal beats.

The blue rectangular boxes are designated with a “B” to distinguish the blue rectangular boxes and the red rectangular boxes when FIGS. 8A and 8B are not available in color.

FIG. 8B illustrates beats employed as templates for a matched filter.

### DETAILED DESCRIPTION

Methods and systems are provided to automatically refine a selected region of an electrogram so that the refined region may be used to identify information (e.g., source location) that is more likely to result in a successful treatment of a patient than if the selected region (without being refined) was used to identify information. In some embodiments, an Automatic Refinement of Electrogram Selection (ARES) system accesses a selected region of a patient electrogram collected from a patient and refines the selected region by adjusting the extent of the selected region so that a mapping system or another system uses the refined electrogram to identify more accurate information (e.g., source location, scar tissue, or area of reentrant circuit) than would be identified if the selected region was used. The ARES system thus effects a particular treatment such as an ablation based on a refined region. The ARES system may also automatically output the more accurate information (e.g., source location) to medical devices such as a robotic magnetic navigation device for guiding a catheter during an ablation procedure. The ARES system may interface with a medical device to automatically receive a selected region of an electrocardiogram (ECG) from the medical device during an ablation procedure, identify a refined region based on the selected region, identify a source location of an arrhythmia based on the refined region, and send to the medical device the source location. A selected region may be specified manually by a person or automatically by a computing system. Although described primarily in the context of refining regions of a cardiogram, the ARES system may be employed to refine regions representing electrical activity of other electromagnetic sources within a body such as a brain (e.g., electroencephalogram) or gastrointestinal tract (e.g., gastroenterogram) or, more generally, electromagnetic waveforms whose regions are mapped to data such as radar waves with regions or signatures mapped to reflecting object types.

In some embodiments, the ARES system may generate derived-to-base mappings that, for a collection of cardiograms (e.g., electrocardiograms or vectorcardiograms), map derived regions of a cardiogram to a base region (e.g., “ground truth” region) of that cardiogram. A base region may have been used to identify a source location or other information (e.g., arrhythmia type) that was used to inform a successful treatment of a patient or selected by a person (e.g., an EP) as likely to result in a successful treatment. The ARES system may generate the mappings based on clinical data and/or simulated data. The clinical data may be patient cardiograms that are each associated with a selected region used to identify a source location that was the basis of a successful medical treatment (e.g., ablation) for a patient. The selected region of a patient cardiogram may have been selected by a medical provider and input to a mapping system to identify a source location that helped inform medical treatment decisions. Because the medical treatment was successful, the selected region is considered to be the base region for that patient cardiogram. The base regions may also have been selected by a person (e.g., an expert) who, for example, reviews simulated cardiograms and selects base regions that are likely to be used by a mapping system to identify a source location that would result in a successful medical treatment. The base regions of simulated cardiograms may also be automatically selected by a source location mapping system for mapping to source locations. A source location mapping system is described in U.S. Pat. No. 10,860,754 entitled “Calibration of Simulated Cardiograms” and granted on Dec. 8, 2020, which is hereby incorporated by reference.

The ARES system may identify additional base regions from candidate base regions of candidate ECGs (clinical and/or simulated). A candidate base region that is similar to a selected base region (e.g., selected manually) and is arrhythmic is selected as a base region for further processing. Similarity may be determined using a similarity metric such as a matched filter, a Pearson correlation, and a cosine similarity. Candidate base regions whose similarity to a selected base region satisfies a similarity criterion (e.g., >0.75 out of 1.00) is selected as a similar base region. To determine similarity, the ARES system may process the candidate ECGs to identify various ECG landmarks, such as P, Q, R, S, and T peaks. The term “peak” refers to a local maximum (i.e., positive peak) or a local minimum (i.e., negative peak) of an ECG. From the ECG landmarks, the ARES system identifies ECG regions such as QRS complexes, T waves, P waves, Q-T intervals, T-Q intervals, T-P intervals, R-R intervals, and so on. To identify ECG landmarks and ECG regions, the ARES system may employ a Pan-Tompkins algorithm or other algorithms such as those available via open-source software systems. One open-source software algorithm is published in GitHub as described in Makowski, D., Pham, T., Lau, Z. J., Brammer, J. C., Lespinasse, F., Pham, H., Schölzel, C., & Chen, S. A., NeuroKit2: A Python toolbox for neurophysiological signal processing, Behavior Research Methods, 53(4), pp. 1689-1696 (2021). Similarity may be determined based on similarity of characteristics of the landmarks (e.g., QRS integral).

For each similar base region, the ARES system may apply a machine learning (ML) model to determine whether the similar base region is normal or arrhythmic. (See, Pat. App. No. PCT/US23/72854 entitled “Automatic Fibrillation Classification and Identification of Fibrillation Epochs” and filed on Aug. 24, 2023, which is hereby incorporated by reference.) The ARES system may also employ similarity techniques (e.g., as described above) to determine similarity between the similar base regions and known arrhythmic regions to determine with a similar base region is normal or arrhythmic. The similar base regions that are considered arrhythmic are selected as base regions for further processing.

In some embodiments, the ARES system generates the derived regions from the base regions. For each base region and cardiogram from which it was selected, the ARES system generates derived regions from the base cardiogram based on variations in the extent of the base region. For example, the ARES system may generate derived regions by adding positive or negative increments to the start time of that base region and/or adding positive or negative increments to the end time of that base region. If the increments are 0.01 seconds within a range of −0.05 to +0.05 seconds, the ARES system may generate 120 ((11*11)−1) derived regions for each base region. The ARES system maps each derived region to the base region from which it was derived and/or to the source location.

The ARES system employs the derived-to-base mappings to refine a subject region selected from a patient cardiogram collected from a patient. Given the subject region, the ARES system identifies and outputs an indication of a base region based on its similarity to the subject region. The base region is considered to represent the refined subject region. Rather than or in addition to identifying the base region, the ARES system may identify and output other information identified based on the base region, such as the source location to which the base region is mapped. Regions may be considered to be similar or matching based on a similarity criterion being satisfied. For example, a similarity score may be calculated (e.g., a Pearson correlation) and the similarity criterion may be that the similarity score is above a threshold similarity score or is the highest similarity score calculated. As described below, a machine learning (ML) model may be used to identify a base region given a subject region. The ML model may be trained with training data that includes derived regions labeled with base regions. The derived-to-base mappings may also be associated with cardiac characteristics, such as heart geometry, scar tissue areas, conduction velocity, and so on, that may be used to select a base region factoring in the cardiac characteristics of the patient. The ML model may be trained using features derived from such cardiac characteristics. The identification of a base region that factors in patient cardiac characteristics is effectively calibrating the identification to patient. (See, U.S. Pat. No. 10,860,754, § “Calibration of Simulations.”)

In some embodiments, after a base region is identified for a subject region (e.g., using an ML model), the ARES system may identify the derived regions for the base region based on the derived-to-base mappings or may dynamically generate the derived regions by varying the extent of the base region. The ARES system may also identify derived regions by varying the extent of a region selected by a person or selected automatically. For example, a person may select a region of a cardiogram, and derived portions may be identified by varying the extent of the selected region. Such derived regions may be used, for example, to identify a source location for each derived region and provide the source locations to an EP to help inform a treatment decision for the patient.

The ARES system may provide a user interface that outputs an indication of the source locations associated with derived regions and/or multiple base regions (e.g., when a subject region is mapped to multiple base regions), for example, by displaying a graphic of a heart indicating the source locations on the graphic to help inform treatment decisions for the patient. For example, if most of the source locations are close to each other, those source locations may be an appropriate treatment target. The ARES system may allow for the selection (e.g., by a user or a computing device) of a subset of the derived regions and output information for that subset. The ARES system may output an indication of the derived region used to select a source location. For example, the ARES system may highlight the derived region of the patient cardiogram and display a graphic of a heart illustrating the source location identified based on the highlighted derived region. A user may select different derived regions from a list of derived regions, and the list may be automatically scrolled to show automatic updates of the highlighted derived region and the source location identified using that derived region on a graphic of a heart. The ARES system may output an indication of the source location to a device the controls treating the patient. For example, the device may be a stereotactic ablative radiotherapy (SABR) device or other device that coordinates performing of a therapy. Alternatively, the ARES system may output only a source location associated with that base region.

In some embodiments, the ARES system may process multiple leads of a cardiogram to identify a source location. The ARES system may have lead mappings for each lead that map lead derived regions of that lead to base regions for that lead. The lead mappings may be generated from clinical data and/or simulated data as described above more generally for a cardiogram. When a region is selected, for example, for one lead, the ARES system may identify a lead selected region for each lead that corresponds to the selected region. For each lead selected region, the ARES system identifies a corresponding lead base region for that lead and a lead source location for that lead from derived-to-base mappings and/or an ML model for that lead. The ARES system then outputs an indication of the lead source locations for a lead (or multiple leads), for example, on a graphic of a heart. Again, if most of the source locations are close to each other, those source locations may be an appropriate treatment target. The ARES system may also rank the source locations (e.g., for a single lead or multiple leads) based, for example, on clusters of similar source locations or generate a combined source location based, for example, on a weighting of the source locations from the average of the source locations. The ranking and the combined source location may be displayed on a graphic of a heart.

In some embodiments, the ARES system may automatically select a region using various techniques. For example, the ARES system may select a region relating to a T-Q interval of an atrial fibrillation (e.g., an AF epoch), a region relating to a ventricular fibrillation (e.g., a VF epoch), a region based on a non-standard QRS complex (e.g., relating to a ventricular tachycardia), and so on. Techniques for automatically selecting an AF epoch and a VF epoch are described in U.S. Pat. App. No. PCT/US23/72854. A non-standard QRS complex may have, for example, abnormal Q and/or S waves. The ARES system may identify the highest peak within a cardiac cycle (e.g., heartbeat), which may correspond to an R peak. The ARES system then identifies the start and the end of the R wave as representing the non-standard QRS complex. To identify the R wave, the ARES system may employ a gradient descent search of an equation defined by the voltage-time series of a cardiogram to identify a valley to the left of the R peak and a valley to the right of the R peak. The lowest points in the valleys may be selected as the start and end of the R wave. Ideally, the lowest point of a valley will have a slope of zero. However, because of noise and resolution of the cardiogram, there may be no point that corresponds to a slope of exactly zero. Thus, the ARES system may select a point with a non-zero slope, for example, a point corresponding to the lowest slope in a valley or a point that has a slope that is within a delta of zero (e.g., +0.05). In addition, the gradient descent search may encounter valleys corresponding to local minimums as it searches down a slope of the R wave. To avoid selecting a start and an end based on the valley of a local minimum, the ARES system may identify the end of a valley and apply a local minimum criterion to determine if it is a local minimum. For example, the local minimum criterion may be based on steepness of the valley, width of the valley, value of the lowest point in the valley (e.g., positive), and so on. If the local minimum criterion is satisfied, the ARES system continues the search from the end of the valley (e.g., at the peak at the end of the valley).

In some embodiments, the ARES system may display a subject cardiogram (e.g., a patient or simulated cardiogram) so that a medical provider can select a subject region. For example, the ARES system may display vertical bars that may be positioned by the medical provider to demarcate the subject start time and the subject end time of the subject region. As the medical provider moves a vertical bar to adjust the subject region, the ARES system may display an indication of the source location associated with each adjusted subject region, for example, on a graphic of a heart. The ARES system may also display a demarcation of the associated base region or other designated region (e.g., the region that is initially displayed), for example, to provide feedback to the medical provider.

In some embodiments, the ARES system may provide a user interface to allow a user to review and override the designation of a region of a cardiogram that may be designated automatically by a computing system, manually by a person, or semi-automatically as described in U.S. Pat. App. No. PCT/US23/72854. Given one or more designated regions of a cardiogram, the ARES system displays the cardiogram with any designated region highlighted (e.g., from the start time to the end time of a designated region). The ARES system may display one lead, multiple leads separately, or multiple leads that are superimposed. A user may scroll to portions of a cardiogram that are not currently visible, for example, by moving a cursor to the left or right of the displayed cardiogram, selecting a left or right scroll button, and so on. The ARES system may highlight each designated region with a rectangular box with a width that covers the start time and the end time and a height that is based on the maximum voltage and the minimum voltage of the cardiogram or each designated region. The highlighting may vary based on distance of the designated region to the current cursor position. For example, when the cursor is positioned within a designated region, the highlight of the designated region may be emphasized (e.g., dark) and the highlight of other designated regions may be deemphasized such as by growing fainter (e.g., more transparent) based on their distance from the cursor. By varying the emphasis, the user may more readily focus on the designated region closest to the cursor. The ARES system may also display information of a designated (or a refined) region such as the start time and the end time and whether it has been selected as a submission region to be submitted to a mapping system to help inform treatment decision for a patient.

The ARES system allows the user to adjust the start time and the end time of each designated region. For example, when a user moves the cursor near a designated vertical edge of a designated box highlighting a designated region, a refined vertical edge may be displayed, such as one overlapping that designated vertical edge. As the user moves the refined vertical edge, the ARES system may highlight the area between the designated vertical edge and the refined vertical edge using different highlighting to differentiate a refined box from the designated box. As discussed above, the ARES system may display a graphic of a heart illustrating a source location based on the time range of a refined box. The graphic may also illustrate the source location of other refined or designated regions. When the user is satisfied with the refinement, the user may select the refined region as a submission region to help inform treatment decisions for a patient.

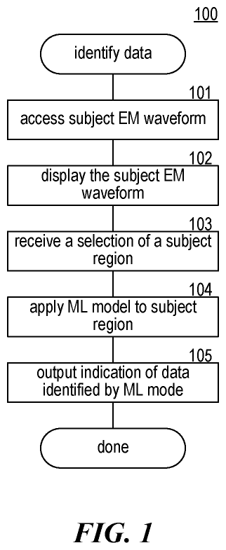

FIG. 1 is a flow diagram that illustrates the processing of an identify data component of the ARES system in some embodiments. The identify data component 100 is provided a subject electromagnetic (EM) waveform (e.g., voltage-time series) and identifies data associated with the subject EM waveform. In block 101, the component accesses the subject EM waveform. In some embodiments, the subject EM waveform is a subject cardiogram collected from the patient and the data is a source location. In block 102, the component displays the subject EM waveform. In block 103, the component receives a selection of a subject region of the subject EM waveform. In block 104, the component applies an ML model to the subject region to identify data associated with the subject region such as a base region and/or a source location of an arrhythmia. The ML model may be trained using mappings of derived regions to the data generated from collected (e.g., clinical) data and/or simulated data. In block 105, the component outputs an indication of the data identified by the ML model and completes.

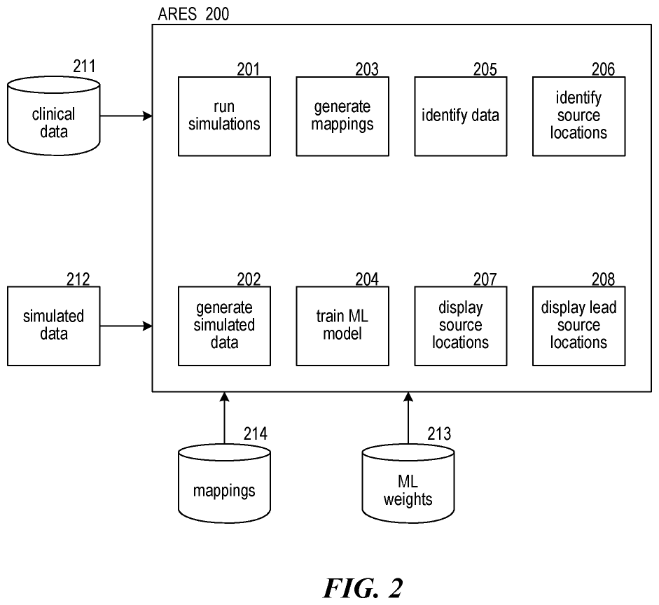

FIG. 2 is a block diagram that illustrates the components of the ARES system in some embodiments. The ARES system 200 includes a run simulations component 201, a generate simulated data component 202, a generate mappings component 203, a train ML model component 204, an identify data component 205, an identify source locations component 206, a display source locations component 207, and a display lead source locations component 208. The ARES system interfaces with a clinical data store 211, a simulated data store 212, an ML weights store 213, and a mappings store 214. The run simulations component may run simulations to simulate electrical activity of the heart based on source locations (or other EM source based on characteristics of that EM source). The generate simulated data component generates simulated base regions from simulated cardiograms derived from the simulated electrical activity and stores associations between the simulated cardiograms, simulated base regions, and simulated source locations in the simulated data store. The generate mappings component generates mappings between derived regions, base regions, and source locations and stores the mappings in the mappings store. The train ML model component trains an ML model to input a subject region and output a source location and/or a base region. The train ML model component stores the weights that are learned in the ML weights store. The identify data component is described above in reference to FIG. 1. The identify source locations component inputs a subject region and outputs a source location. The display source locations component displays the source locations associated with derived subject regions. The display lead source locations component displays a source location associated with each lead of a cardiogram.

The mappings store may employ various data structure architectures. For example, the data structure may be a derived-to-base table that includes an entry for each derived region that is mapped to its base region. In such a case, a patient region may be compared to each derived region to identify the most similar derived region and to select that base region to which is mapped to. However, if the number of derived regions is large, such comparison may have a high time complexity. To reduce the time complexity, the ARES system may generate clusters of similar derived regions using, for example, a k-means clustering technique to generate some number of clusters (e.g., 100) that is each associated with a mean derived region. To identify a base region for a patient region, the ARES system identifies the cluster whose mean is most similar to the patient region. The ARES system may then identify the derived region of that cluster that is most similar and then select the base region from the derived-to-base table that is mapped to that derived region. Other data structures may be employed such as a hash table that clusters together derived regions with the same hash code generated by a hash function that generates one of, for example, 100 possible hash codes.

The computing systems (e.g., network nodes or collections of network nodes) on which the ARES system and the other described systems may be implemented may include a central processing unit, input devices, output devices (e.g., display devices and speakers), storage devices (e.g., memory and disk drives), network interfaces, graphics processing units, communications links (e.g., Ethernet, Wi-Fi, cellular, and Bluetooth), global positioning system devices, and so on. The input devices may include keyboards, pointing devices, touch screens, gesture recognition devices (e.g., for air gestures), head and eye tracking devices, microphones for voice recognition, and so on. The computing systems may include high-performance computing systems, distributed systems, cloud-based computing systems, client computing systems that interact with cloud-based computing system, desktop computers, laptops, tablets, e-readers, personal digital assistants, smartphones, gaming devices, servers, and so on. The computing systems may access computer-readable media that include computer-readable storage mediums and data transmission mediums. The computer-readable storage mediums are tangible storage means that do not include a transitory, propagating signal. Examples of computer-readable storage mediums include memory such as primary memory, cache memory, and secondary memory (e.g., DVD), and other storage. The computer-readable storage media may have recorded on them or may be encoded with computer-executable instructions or logic that implements the ARES system and the other described systems. The data transmission media are used for transmitting data via transitory, propagating signals or carrier waves (e.g., electromagnetism) via a wired or wireless connection. The computing systems may include a secure crypto processor as part of a central processing unit (e.g., Intel Secure Guard Extension (SGX)) for generating and securely storing keys and for encrypting and decrypting data using the keys and for securely executing all or some of the computer-executable instructions of the ARES system. Some of the data sent by and received by the ARES system may be encrypted, for example, to preserve patient privacy (e.g., to comply with government regulations such the European General Data Protection Regulation (GDPR) or the Health Insurance Portability and Accountability Act (HIPAA) of the United States). The ARES system may employ asymmetric encryption (e.g., using private and public keys of the Rivest-Shamir-Adleman (RSA) standard) or symmetric encryption (e.g., using a symmetric key of the Advanced Encryption Standard (AES)).

The one or more computing systems may include client-side computing systems and cloud-based computing systems (e.g., public or private) that each executes computer-executable instructions of the ARES system. A client-side computing system may send data to and receive data from one or more servers of the cloud-based computing systems of one or more cloud data centers. For example, a client-side computing system may send a request to a cloud-based computing system to perform tasks such as run a patient-specific simulation of electrical activity of a heart or train a patient-specific machine learning model. A cloud-based computing system may respond to the request by sending to the client-side computing system data derived from performing the task such as a source location of an arrhythmia. The servers may perform computationally expensive tasks in advance of processing by a client-side computing system such as training a machine learning model or in response to data received from a client-side computing system. A client-side computing system may provide a user experience (e.g., user interface) to a user of the ARES system. The user experience may originate from a client computing device or a server computing device. For example, a client computing device may generate a patient-specific graphic of a heart and display the graphic. Alternatively, a cloud-based computing system may generate the graphic (e.g., in a Hyper-Text Markup Language (HTML) format or an extensible Markup Language (XML) format) and provide it to the client-side computing system for display. A client-side computing system may also send data to and receive data from various medical devices such as an ECG monitor, an ablation therapy device, an ablation planning device, and so on. The data received from the medical devices may include an ECG, actual ablation characteristics (e.g., ablation location and ablation pattern), and so on. The data sent to a medical device may be, for example, in a Digital Imaging and Communications in Medicine (DICOM) format. A client-side computing device may also send data to and receive data from medical computing systems that store patient medical history data (e.g., an electronic health record (EHR) system), descriptions of medical devices (e.g., type, manufacturer, and model number) of a medical facility, that store, medical facility device descriptions, that store results of procedures, and so on. The term cloud-based computing system may encompass computing systems of a public cloud data center provided by a cloud provider (e.g., Azure provided by Microsoft Corporation or Amazon Web Services (AW) provided by Amazon.com, Inc.) or computing systems of a private server farm (e.g., operated by the provider of the ARES system.

The ARES system and the other described systems may be described in the general context of computer-executable instructions, such as program modules and components, executed by one or more computers, processors, or other devices. Program modules or components include routines, programs, objects, data structures, and so on that perform tasks or implement data types of the ARES system and the other described systems. Typically, the functionality of the program modules may be combined or distributed as desired. Aspects of the ARES system and the other described systems may be implemented in hardware using, for example, an application-specific integrated circuit (ASIC) or a field programmable gate array (FPGA).

A machine learning (ML) model used by the ARES system may be any of a variety or combination of supervised, semi-supervised, self-supervised, unsupervised, or reinforcement learning ML models including a neural network such as fully connected, convolutional, recurrent, or autoencoder neural network, transformer, autoencoder, and so on. A supervised ML model for the ARES system is trained using training data that includes features derived from data and labels corresponding to the data. For example, the data may be images derived regions with a feature being the image itself, a time-voltage series and/or features derived from the image or features of the patient (e.g., arrhythmia type), and the labels may be a characteristic indicated by the ECGs (e.g., base region, subject region, or source location). The collection of features is referred to as a feature vector. The training results in a set of weights for the ML model such as weights of activation functions of the layers of a neural network. The trained ML model can then be applied to a feature vector (e.g., derived from a subject region or refined region) to generate a label (e.g., base region) for the feature vector.

A neural network that may be employed by the ARES system has three major components: architecture, loss function, and search algorithm. The architecture defines the functional form relating the inputs to the outputs (in terms of network topology, unit connectivity, and activation functions). The search in weight space for a set of weights that minimizes the loss function is the training process. The loss function is a metric based on the differences between the labels of the training data and the labels generated by the neural network given its current weights. The goal of the training process is to learn weights so that when the neural network is applied to training data the loss function is minimized. A neural network may use a radial basis function (RBF) network and a standard or stochastic gradient descent as the search technique with backpropagation. As described above, the features used in training a neural network may be derived from an ECG (e.g., image or time-voltage series) and may not include, for example, an ECG image or time-voltage series.

A convolutional neural network (CNN) that may be employed by the ARES system has multiple layers such as a convolutional layer, a rectified linear unit (ReLU) layer, a pooling layer, a fully connected (FC) layer, and so on. Some more complex CNNs may have multiple convolutional layers, pooling layers, and FC layers. Each layer includes a neuron for each output of the layer. A neuron inputs outputs of prior layers (or original input) and applies an activation function to the inputs to generate an output. An example of a CNN is based on the U-Net architecture.

A convolutional layer may include multiple filters (also referred to as kernels or activation functions). A filter inputs a convolutional window, for example, of an ECG image, applies weights to each pixel of the convolutional window, and outputs value for that convolutional window. For example, if the static image is 256 by 256 pixels, the convolutional window may be 8 by 8 pixels. The filter may apply a different weight to each of the 64 pixels in a convolutional window to generate the value.

An activation function has a weight for each input and generates an output by combining the inputs based on the weights. The activation function may be a rectified linear unit (ReLU) that sums the values of each input times its weight to generate a weighted value and outputs max (0,weighted value) to ensure that the output is not negative. The weights of the activation functions are learned when training a ML model. The ReLU function of max (0,weighted value) may be represented as a separate ReLU layer with a neuron for each output of the prior layer that inputs that output and applies the ReLU function to generate a corresponding “rectified output.”

A pooling layer may be used to reduce the size of the outputs of the prior layer by downsampling the outputs. For example, each neuron of a pooling layer may input 16 outputs of the prior layer and generate one output resulting in a 16-to-1 reduction in outputs.

The ARES system may employ a CNN based on a U-Net ML model. The U-Net ML model includes a contracting path and an expansive path. The contracting path includes a series of max pooling layers to reduce spatial information of the input image and increase feature information. The expansive path includes a series of upsampling layers to convert the feature information to the output image. The input and output of a U-Net represent an image such as an image of patient ECG as input and an image of a base region as output.

The ARES system may employ multimodal machine learning to combine different modalities of input data to identify a base region or a source location. The modalities may be, for example, images and data derived from electronic heath records (EHRs) such as cardiac characteristics or attributes derived from clinical and/or simulated data (e.g., cardiac hypertrophy, conduction velocity, prior ablation location, and arrythmia type). The ARES system may employ a multimodal ML model to process image data and cardiac characteristics.

In one multimodal ML approach, referred to as “early fusion,” data of the different modalities is combined at the input stage and is then trained on the multimodal data. The training data for these modalities include feature vectors generated from a collection of sets of an image and other features such as arrhythmia type and prior ablation location and labels (e.g., base region) for the feature vectors. The image and other features may be used in its original form or preprocessed, for example, to reduce its dimensionality, for example, applying a principal component analysis. The vectors are labeled with a base region or source location and then used to train an ML model using primarily using supervised approaches although self-supervised or unsupervised approaches may also be used.

In a second multimodal ML approach, data from different modalities may be kept separate at the input stage and used as inputs to different, modality-specific ML models (e.g., a CNN for image data and a neural network for cardiac characteristics). The modality-specific ML models may be trained jointly such that information from across different modalities is combined to make predictions, and the combined (cross-modality) loss is used to adjust model weights. Alternatively, the modality-specific ML models may also be trained separately using a separate loss function for each modality. A combined ML model is then trained based on the outputs of the modality specific models. Continuing with the example, the training data for each modality-specific ML model may be based on its data along with a label. The combined ML model is then trained with the outputs of the modality-specific ML models with a final label such as derived region or source location.

The ARES system may employ as transformed ML model. A transformer ML was introduced as an alternative to a recurrent neural network that is both more effective and more parallelizable. (See, Vaswani, Ashish, et al., “Attention is all you need,” Advances in neural information processing systems 30 (2017), which is hereby incorporated by reference.) Transformer ML was originally described in the context of natural language processing (NLP) but has been adapted to other applications such as image processing to augment or replace a CNN. In the following, transformer is described in the context of NLP as introduced by Vaswani.

A transformer includes an encoder whose output is input to a decoder. The encoder includes an input embedding layer followed by one or more encoder attention layers. The input embedding layer generates an embedding of the inputs. For example, if a transformer ML model is used to process a sentence as described by Vaswani, each word may be represented as a token that includes an embedding of a word and its positional information. Such an embedding is a vector representation of a word such that words with similar meanings are closer in the vector space. The positional information is based on position of the word in the sentence.

The first encoder attention layer inputs the embeddings and the other encoder attention layers input the output from the prior encoder attention layer. An encoder attention layer includes a multi-head attention mechanism followed by a normalization sublayer whose output is input to a feedforward neural network followed by a normalization sublayer. A multi-head attention mechanism includes multiple self-attention mechanisms that each inputs the encodings of the previous layer and weighs the relevance encodings to other encodings. For example, the relevance may be determined by the following attention function:

$$Attention ( Q , K , V ) = soft max ( \frac{{QK}^{T}}{\sqrt{{d}_{k}}} ) V$$

where Q represents a query, K represents a key, V represents a value, and dₖ represents the dimensionality of K. This attention function is referred to as scaled dot-product attention. In Vaswani, the query, key, and value of an encoder multi-head attention mechanism is set to the input of the encoder attention layer. The multi-head attention mechanism determines the multi-head attention as represented by the following:

Multillead(Q,K,V)=concat(head₁, . . . ,head₈)W^{o}

headᵢ=Attention(QWᵢ^{Q},KWᵢ^{K},VWᵢ^{V})

where W represents weights that are learned during training. The weights for the feedforward networks are also learned during training. The weights may be initialized to random values. A normalization layer normalizes its input to a vector having a dimension as expected by the next layer or sub-layer.

The decoder includes an output embedding layer, decoder attention layer, a linear layer, and a softmax layer. The output embedding layer inputs the output of the decoder shifted right. Each decoder attention layer inputs the output of the prior decoder attention layer (or the output embedding layer) and the output of the encoder. The embedding layer is input to the decoder attention layer, the output of the decoder attention layer is input the linear layer, and the output of the linear layer is input to the softmax layer which outputs probabilities. A decoder attention layer includes a decoder masked multi-head attention mechanism followed by a normalization sublayer, a decoder multi-head attention mechanism followed by a normalization sublayer, and a feedforward neural network followed by a normalization sublayer. The decoder masked multi-head attention mechanism masks the input so that predictions for a position are only based on outputs for prior positions. A decoder multi-head attention mechanism inputs the normalized output of the decoder masked multi-head attention mechanism as a query and the output of the encoder as a key and a value. The feedforward neural network inputs the normalized output of the decoder multi-head attention mechanism. The normalized output of the feedforward neural network is the output of that multi-head attention layer. The weights of the linear layer are also learned during training.

After being trained, a sentence may be input to encoder to generate an encoding of the sentence that is input to the decoder. Initially, the output of the decoder that is input to the decoder is set to null. The decoder then generates an output based on the encoding and the null input. The output of the decoder is appended to the decoder's current input, and the decoder generates a new output. This decoding process is repeated until the encoder generates a termination symbol. If the transformer is trained with English sentences labeled with French sentences, then a termination symbol is added to the end of the French sentences. When translating a sentence, the transformer terminates its translation when the termination symbol is generated indicating the end of the French sentence that is completion of the translation.

Although initially developed to process sentences, transformers have been adapted for image recognition. The input a decoder of a transformer may be a representation of fixed-size patches of the image. (See, Dosovitskiy, et al., “An Image is worth 16×16 Words: Transformers for image Recognition at Scale,” arXiv: 2020-11929, Jun. 3, 2021, which is hereby incorporated by reference.) The representation of a patch may be, for each pixel of the patch, an encoding of its row, column, and color. The output of the encoder is fed into neural network to generate a classification of the image.

The ARES system may employ an encoder of a transformer and a neural network to generate a base region or source location given a subject region. The encoder inputs tokens (e.g., 16×16 pixels of an image or portions of a time-voltage series) of the subject region and generates an encoding that is input into a neural network that generates the base region or source location. The neural network may also input patient cardiac characteristics. The encoder and neural network are trained with a combined loss function.

As discussed above, the ARES system may employ ML models that input a cardiogram input a feature vector of one or more features derived from the cardiogram. The features may include an image of cardiogram, a time-voltage series specifying voltages and time increments of the cardiogram, images and time-voltage series of portions of the cardiogram (e.g., QRS complex), length in seconds of various intervals (e.g., R-R interval, QRS complex, T wave, T-Q interval, and Q-R interval), QRS integral, maximum, minimum, mean, and variance of voltages of portions of the cardiogram, a maximal vector of QRS loop and angle of the vector derived from VCG, location of a peak (Q peak) or zero crossing relative to a maximum peak (T peak) in an interval, and so on. The features used by an ML model may be manually or automatically selected. An assessment of which features may be useful in providing an accurate output for a ML model are referred to as informative feature. The assessment of which features are informative may be based on various feature selection techniques such as a predictive power score, a lasso regression, a mutual information analysis, and so on.

The features may also be latent vectors generated using a ML model such as an autoencoder. For example, an autoencoder may be trained using ECG images. In such a case, when an ECG image is input into the trained autoencoder, the latent vector that is generated is a feature vector that represents the ECG image. That feature vector can be input into another trained ML model such as a neural network or support vector machine to generate an output. When training the other ML model, for example, to output a base region, a refined region, a source location, or an arrhythmia type, the training ECG images are input to the autoencoder to generate training feature vectors that are labeled with a base region, a refined region, a source location, or an arrhythmia type. The other ML model is then trained using the labeled feature vectors. The autoencoder may be trained using the training ECG images or may have been previously trained using a collection of ECG images. Rather than pre-training an autoencoder, only the portion of the autoencoder that generates the latent vector may be trained in parallel with the other ML model using a combined loss function. In such a case, no autoencoding is performed. Rather the latent vector represents features of an ECG image that are particularly relevant to generating the output of the other ML model. Such an ML architecture may be used, for example, when the other ML model (e.g., transformer) is not designed to process ECG images directly.

The ARES system may employ an unsupervised ML technique to train an ML model using derived regions with base region labels. K-means clustering is an example ML technique. Given feature vectors representing the derived regions, k-means clustering clusters the feature vectors into cluster of similar feature vectors. With k-means clustering, the number of clusters may be predefined. For example, the classification system may employ three clusters (k=50) to represent a cluster of derived regions. An example training technique initially randomly places a feature vector in each cluster. The training then repeatedly calculates a mean feature vector of each cluster, selects a feature vector not in a cluster, identifies the cluster whose mean is most similar, adds the feature vector to that cluster, and moves the feature vectors already in the clusters to the cluster with the most similar mean. Similarity may be determined, for example, based on a Pearson similarity, a cosine similarity, and so on. The training ends when all the feature vectors have been added to a cluster. Each derived region in a cluster can be mapped to its base region. Alternatively, an average base region may be generated for each cluster.

In some embodiments, the ARES system may employ a kNN model. The training data for a kNN model may be training feature vectors based on derived regions and labeled with base regions. A kNN model may be used without a training phase that is without learning weights or other parameters to represent the training data. In such a case, subject region is compared to the training feature vectors to identify a number (e.g., represented by the “k” in kNN) of similar training feature vectors. Once the number of similar training feature vectors are identified, the labels associated with the similar training feature vectors are analyzed to generate a base region. The labels of the training feature vectors that are more similar to a subject region feature vector may be given a higher weight than those that are less similar. For example, if k is 10 and four training feature vectors are very similar and six are less similar, similarity weights of 0.9 may be assigned to the very similar training feature vectors and 0.2 to the less similar. If three of the four and one of the six have approximately the same derived region, then the base region is primarily based on those four even though most of the 10 have different information. Conceptually, training feature vectors that are very similar are closer to a feature vector derived from a base region in a multi-dimensional space of features and a similarity weight is based on distance between the feature vectors. Various techniques may be employed to calculate a similarity metric indicating similarity between a candidate feature vector and a training feature vector such as a dot product, cosine similarity, a Pearson's correlation, and so on.

If the number of training feature vectors is large, various techniques may be employed to effectively “compress” the training data during a training phase. For example, a clustering technique may be employed to identify clusters of training feature vectors that are similar and have the same label. A training feature vector may be generated for each cluster (e.g., one from the cluster or one based on mean values for the features) as a cluster feature vector and assign a cluster weight to it based on number of training feature vectors in the cluster.

The ML models that input a cardiogram input a feature vector of one or more features derived from the cardiogram. The features may include an image of cardiogram, a time-voltage series specifying voltages and time increments of the cardiogram, images and time-voltage series of portions of the cardiogram (e.g., QRS complex), length in seconds of various intervals (e.g., R-R interval, QRS complex, T wave, T-Q interval, and Q-R interval), QRS integral, maximum, minimum, mean, and variance of voltages of portions of the cardiogram, a maximal vector of QRS loop and angle of the vector derived from VCG, location of a peak (Q peak) or zero crossing relative to a maximum peak (T peak) in an interval, and so on. The features used by an ML model may be manually or automatically selected. An assessment of which features may be useful in providing an accurate output for a ML model are referred to as informative feature. The assessment of which features are informative may be based on various feature selection techniques such as a predictive power score, a lasso regression, a mutual information analysis, and so on.

The features may also be latent vectors generated using a ML model such as an autoencoder. For example, a CNN autoencoder may be trained using ECG images of derived regions. In such a case, when an ECG image is input into the trained autoencoder, the latent vector that is generated is a feature vector that represents the ECG image. That feature vector can be input into another trained ML model such as a neural network or support vector machine to generate an output. When training a ML model, for example, to classify an ECG as representing an atrial fibrillation or a ventricular fibrillation, the training ECG images are input to the autoencoder to generate training feature vectors that are labeled as being atrial fibrillation or ventricular fibrillation. The other ML model is then trained using the labeled feature vectors. The autoencoder may be trained using the training ECG images or may have been previously trained using a collection of ECG images. Rather pre-training an autoencoder, only the portion of the autoencoder that generates the latent vector may be trained in parallel with the other ML model using a combined loss function. In such a case, no autoencoding is performed. Rather the latent vector represents features of an ECG image that are particularly relevant to generating the output of the other ML model. Such an ML architecture may be used, for example, when the other ML model (e.g., transformer) is not designed to process ECG images directly.

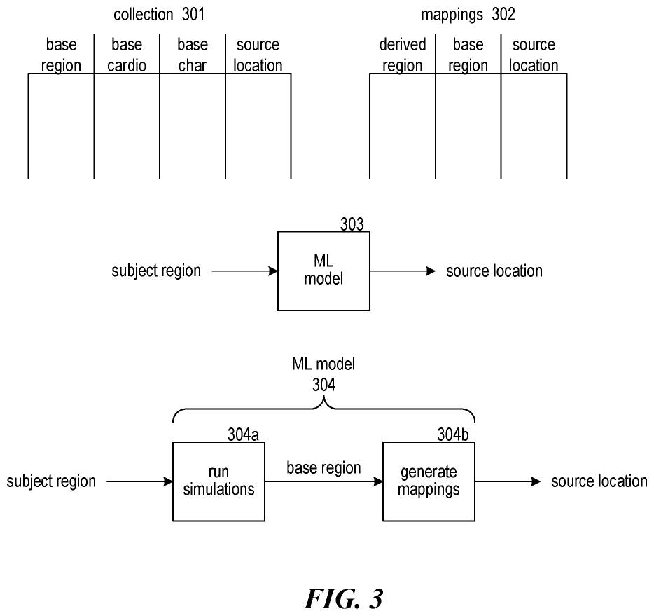

FIG. 3 illustrates some data structures and machine learning architectures of the ARES system in some embodiments. The collection 301 stores clinical data and/or simulated data that contain associations between base regions, base cardiograms, base characteristics, and source locations. The mappings 302 map derived regions to base regions and source locations. The ML model 303 inputs a subject region and outputs a source location. The ML model 304 includes an ML sub-model 304a and an ML sub-model 304b. The ML sub-model (region ML model) 304a inputs a subject region and outputs a base region. The ML sub-model (source location ML model) 304b inputs a base region and outputs a source location. When processing multiple leads, a separate lead ML model may be trained for each lead. Alternatively, a lead ML model may input multiple leads and output a source location. The models and sub-models may be, for example, convolutional neural networks that process an image of the subject region, neural networks or recurrent neural networks that input a voltage-time series, and so on. The model weights may be learned using a loss (or an objective) function based on a measure of the differences between the labels and the outputs. The ML sub-models may be trained separately with their own loss function or trained in parallel with a combined loss function. A gradient descent technique may be used to guide the setting of weights that tend to minimize the difference.

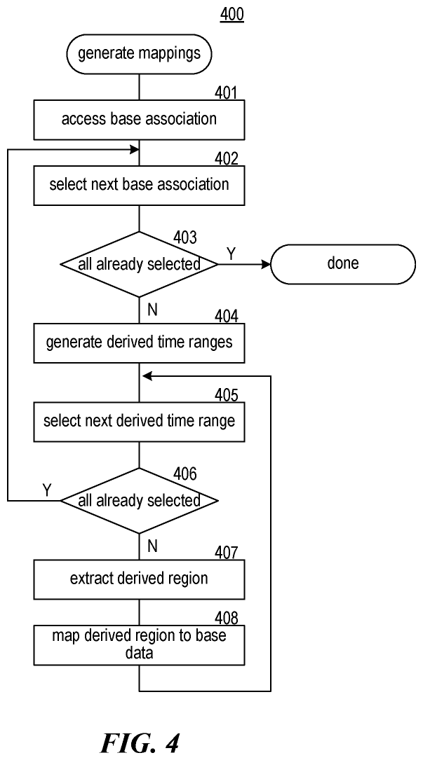

FIG. 4 is a flow diagram that illustrates the processing of the generate mappings component of the ARES system. The generate mappings component 400 is invoked to generate mappings based on clinical data and/or simulated data. In block 401, the component accesses the associations of the clinical data and/or simulated data (e.g., base regions to source locations). In block 402, the component selects the next base association. In decision block 403, if all the base associations have already been selected, then the component completes, else the component continues at block 404. In block 404, the component generates the derived time ranges by adding positive or negative increments to the base region of the association. In block 405, the component selects the next derived time range. In decision block 406, if all the derived time ranges have already been selected, then the component loops to block 402 to select the next base association, else the component continues at block 407. In block 407, the component extracts the derived region that spans the derived time range. In block 408, the component generates a mapping of the derived region to base data such as the base region and/or the source location of the association and then loops to block 405 to select the next derived time range.

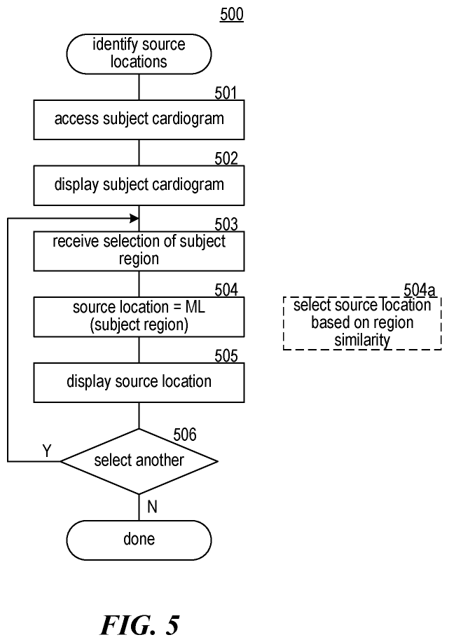

FIG. 5 is a flow diagram that illustrates the processing of an identify source locations components of the ARES system in some embodiments. The identify source locations component 500 is invoked to identify a source location. In block 501, the component accesses a subject cardiogram that has been collected from a patient. The component may be adapted to interface with a device that controls the collecting of the cardiogram. In block 502, the component displays the subject cardiogram. In block 503, the component receives a selection of a subject region such as by a medical provider moving vertical bars to demarcate a start time and an end time. In block 504, the component may invoke an ML model to identify a source location given the subject region. Alternatively, in block 504a, the component may select a source location based on region similarity to a derived region of the derived-to-base mappings. In block 505, the component displays the source location, which may be superimposed on a graphic of a heart. In decision block 506, if another subject region is to be selected, then the component loops to block 503, else the component completes.

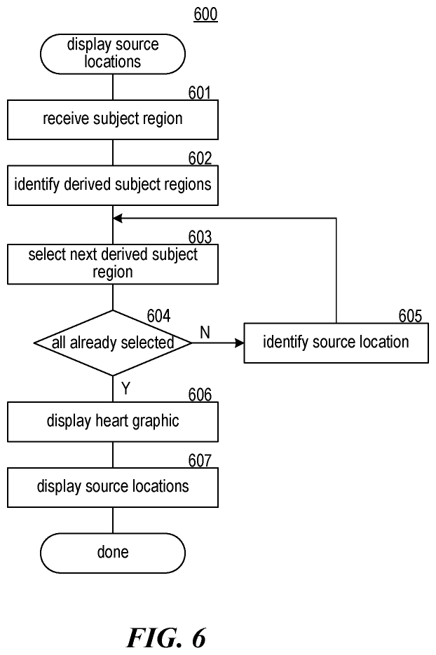

FIG. 6 is a flow diagram that illustrates the processing of a display source locations component of the ARES system in some embodiments. The display source locations component 600 is invoked to display source locations for multiple derived subject regions. In block 601, the component receives a subject region. In block 602, the component identifies derived subject regions by, for example, adjusting the start time and the end time by various increments. In block 603, the component selects the next derived subject region. In decision block 604, if all the derived subject regions have already been selected, then the component continues at block 606, else the component continues at block 605. In block 605, the component identifies the source location associated with the derived subject region, for example, by submitting the derived subject region to a source location mapping system, and then loops to block 603 to select the next derived subject region. In block 606, the component displays a graphic of the heart. In block 607, the component displays an indication of the source locations superimposed on the graphic of the heart and then completes.

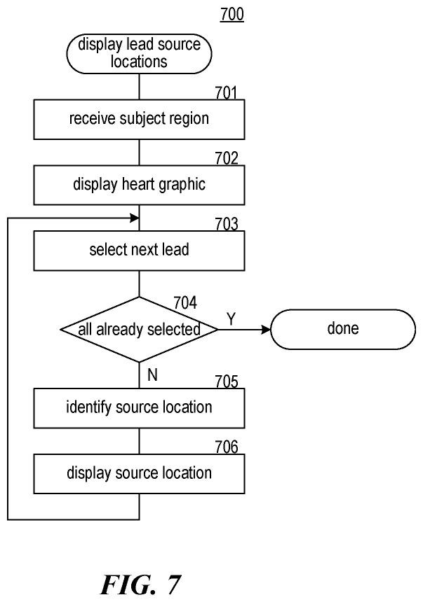

FIG. 7 is a flow diagram that illustrates the processing of a display lead source locations component of the ARES system in some embodiments. The display lead source locations component 700 is invoked to display the source locations associated with multiple leads of a cardiogram. In block 701, the component receives a specification of the subject region within a subject cardiogram. In block 702, the component displays a graphic of a heart. In block 703, the component selects the next lead of the subject cardiogram. In decision block 704, if all the leads have already been selected, then the component completes, else the component continues at block 705. In block 705, the component identifies a source location associated with the lead, for example, by applying an ML model trained using the mappings to the subject region of the lead or by submitting subject region to a source location mapping system. In block 706, the component displays the source location superimposed on a graphic of the heart and then loops to block 703 to select the next lead.

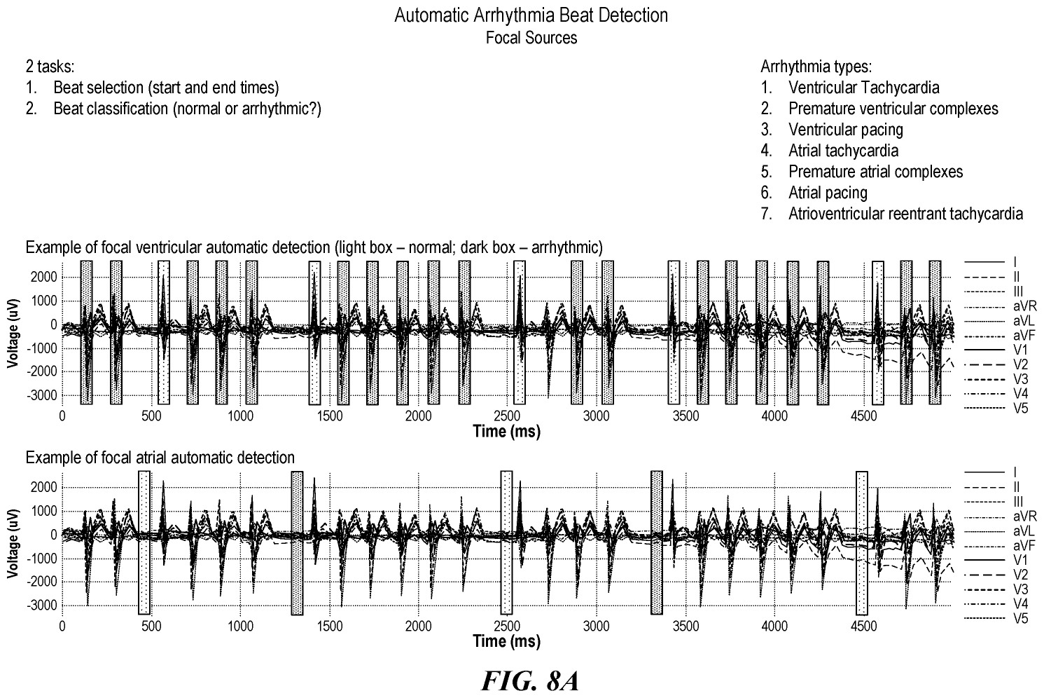

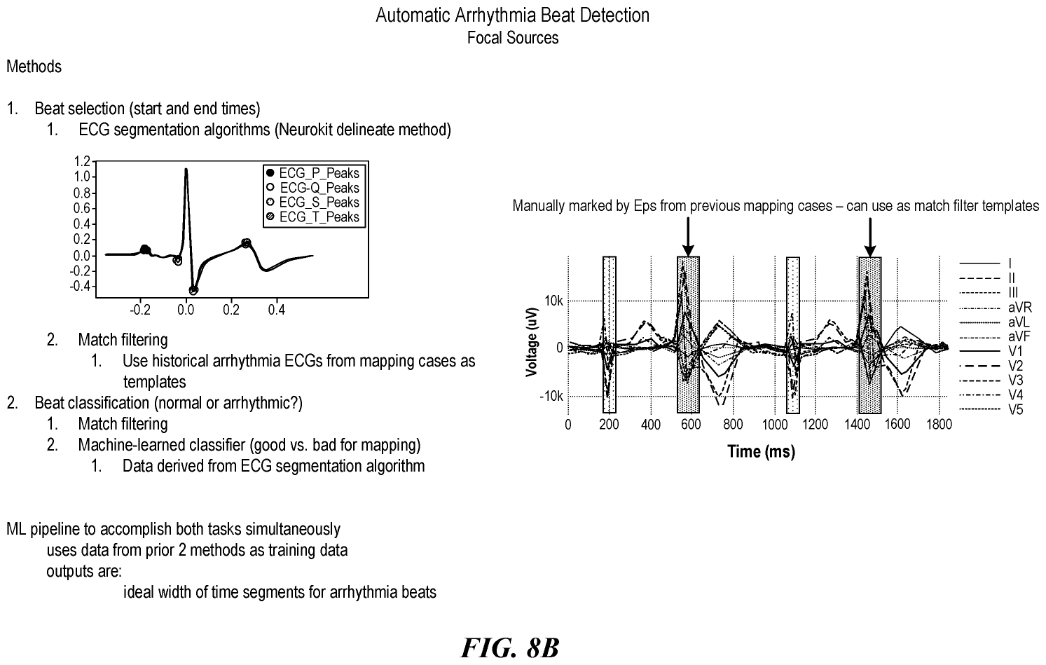

FIG. 8 illustrates examples of automatically detected arrhythmic and normal beats. FIG. 8B illustrates beats employed as templates for a matched filter. The blue rectangular boxes represent normal beats, and the red rectangular boxes represent abnormal beats. By selecting the vertical side of a box, a user may specify the time range of a selected region. The blue rectangular boxes are designated with a “B” to distinguish the blue rectangular boxes and the red rectangular boxes when FIGS. 8A and 8B are not available in color. A beat (i.e., a cycle) representing a region may be automatically detected using an ECG segmentation algorithm based on identification of ECG landmarks or using a matched filter algorithm using, for example, base regions or manually demarcated regions as templates. The beat can then be classified using various techniques such as those described in U.S. Pat. App. No. PCT/US23/72854, by employing a matched filter with arrhythmia beats as templates, or a ML model that is trained using beats labeled as arrhythmia beats or normal beats. The ARES system may also be employed to identify a base region given a manual selection of a beat and use that beat to identify a source location or other data of interest.

In some embodiments, the ARES system may be adapted to derive a time-voltage series of an ECG from an image of an ECG such as a scan of a printed ECG, picture taken of a printed ECG, and so on. Techniques for generating such time-voltage series are described in PCT App. No. PCT/US23/22146 entitled “Encoding Electrocardiogramata” and filed on May 12, 2023, which is hereby incorporated by reference. A time-voltage series of an ECG may also be received from a medical device or EHR and may be in a DICOM format or another standard format.

The following paragraphs describe various aspects of the ARES system. An implementation of the ARES system may employ any combination or sub-combination of the aspects and may employ additional aspects. The processing of the aspects may be performed by one or more computing systems with one or more processors that execute computer-executable instructions that implement the aspects and that are stored on one or more computer-readable storage mediums.

In some aspects, the techniques described herein relate to a method performed by one or more computing systems for training a machine learning model (ML) for identifying data associated with a region of a cardiogram, the method including: accessing a plurality of mappings that each map a base region of a base cardiogram to base data; for each of the plurality of mappings, deriving a plurality of derived regions from the base region and the base cardiogram of that mapping; and for each of the plurality of the derived regions, generating training data that includes the derived regions labeled with the base data of that mapping; and training the ML model using the training data wherein the trained ML model, when applied to a subject region of a subject cardiogram, outputs base data as subject data for the subject region.

In some aspects, the techniques described herein relate to a method wherein a base region of a base cardiogram has a base start time and a base end time within that base cardiogram and wherein the derived regions that are derived from a base region have derived start times and derived end times that are derived from the base start time and the base end time of that base region.

In some aspects, the techniques described herein relate to a method wherein the derived start times and the derived end times for derived regions that are derived from a base region are derived by adding a positive or negative increment to the base start time of that base region and/or adding a positive or negative increment to the base end time of that base region. In some aspects, the techniques described herein relate to a method further including: receiving a subject region of a subject cardiogram; and applying the trained ML model to the subject region, which outputs subject data. In some aspects, the techniques described herein relate to a method wherein the ML model includes a convolutional neural network that inputs an image of a derived region and outputs the base data. In some aspects, the techniques described herein relate to a method wherein the ML model includes a portion of autoencoder that generates a latent vector representing an image of the derived region and another ML model that inputs the generated latent vector and outputs the base data. In some aspects, the techniques described herein relate to a method wherein the ML model is trained using features derived from the derived region the features being one or more of a time-voltage series specifying voltages and time increments of the derived region, images and time-voltage series of portions of the derived region (e.g., QRS complex), length in seconds of various intervals (e.g., R-R interval, QRS complex, T wave, T-Q interval, and Q-R interval) of the derived region, QRS integral of the derived region, maximum, minimum, mean, and variance of voltages of the derived region, a maximal vector of QRS loop and angle of a vector derived from vectorcardiogram of the derived region, and location of a peak (Q peak) or zero crossing relative to a maximum peak (T peak) in an interval of the derived region. In some aspects, the techniques described herein relate to a method wherein the ML model includes an encoder of a transformer adapted to processes images and a neural network inputs the encoding and other features of a feature vector based on the training data. In some aspects, the techniques described herein relate to a method wherein the ML model includes a neural network that inputs a feature vector representing the derived region. In some aspects, the techniques described herein relate to a method wherein the feature vector representing the derived region includes a time-voltage series of the derived region. In some aspects, the techniques described herein relate to a method wherein at least some of the mappings have a base time range of the base cardiogram that is selected by a person and have base data that is specified based on treatment of an arrhythmia. In some aspects, the techniques described herein relate to a method wherein a base cardiogram includes multiple leads and wherein a base region of each lead is mapped to base data. In some aspects, the techniques described herein relate to a method wherein a machine learning model is trained for each lead and further including: receiving lead subject regions of multiple leads of a subject cardiogram; for each of the multiple leads, applying a trained machine learning model for that lead to the lead subject region of that lead, which outputs lead subject data for that lead; and determining overall subject data based on analysis of the lead subject data for the leads. In some aspects, the techniques described herein relate to a method wherein the data is a source location of an arrhythmia. In some aspects, the techniques described herein relate to a method wherein the base data is a region of a cardiogram. In some aspects, the techniques described herein relate to a method wherein the data is a start time and an end time of a region a cardiogram. In some aspects, the techniques described herein relate to a method wherein the derived region of the training data is specified by at least a portion of the base cardiogram of the derived region and a derived start time and a derived end time of the derived region within the base cardiogram.

In some aspects, the techniques described herein relate to a method performed by one or more computing systems for identifying a base region of a cardiogram to inform a treatment decision for a patient, the method including: accessing a subject region of a subject cardiogram collected from the patient; identifying a subject base region of the subject cardiogram based on mappings of derived regions of base cardiograms to base regions of base cardiograms, each derived region being derived from the base region of a base cardiogram to which the derived region is mapped; and outputting an indication of the subject base region of the subject cardiogram to inform a treatment decision for a patient. In some aspects, the techniques described herein relate to a method wherein at least some of the base regions were used in identifying a source location that resulted in successful treatment of an arrhythmia. In some aspects, the techniques described herein relate to a method further including identifying a subject source location of an arrhythmia based on the subject region. In some aspects, the techniques described herein relate to a method wherein the identifying of the subject source location includes inputting the subject base region to a machine learning model that outputs the subject source location. In some aspects, the techniques described herein relate to a method wherein the identifying of the subject base region is based on similarity between the subject base region and the derived regions. In some aspects, the techniques described herein relate to a method wherein the identifying of the subject base region includes inputting the subject base region to a machine learning model that outputs the subject base region. In some aspects, the techniques described herein relate to a method wherein the machine learning model is trained based on the mappings. In some aspects, the techniques described herein relate to a method wherein a base region of a base cardiogram has a base start time and a base end time within that base cardiogram and wherein the derived regions that are derived from a base cardiogram have derived start times and derived end times that are derived from the base start time and the base end time of that base region. In some aspects, the techniques described herein relate to a method wherein the derived start times and the derived end times for derived regions that are derived from a base region are derived by adding positive or negative increments to the base start time of that base region and/or adding positive or negative increments to the base end time of that base region. In some aspects, the techniques described herein relate to a method wherein a region is specified as a time range within a cardiogram, as a voltage-time series, or as an image of a portion of a cardiogram. In some aspects, the techniques described herein relate to a method further including displaying the subject cardiogram, receiving a selection of the subject region of the displayed subject cardiogram, and displaying an indication of the subject region on the displayed subject cardiogram. In some aspects, the techniques described herein relate to a method further including displaying an indication of the subject base region on the displayed subject cardiogram.

In some aspects, the techniques described herein relate to a method performed by one or more computing systems for identifying a source location of an arrhythmia of a patient, the method including: displaying a subject cardiogram of the patient that is collected during an arrhythmia episode of the patient; receiving a selection by a user of a subject region of the subject cardiogram; applying a region machine learning model to the subject region to identify a subject base region of the subject cardiogram; applying a source location machine learning model to the subject base region to identify a subject source location; and outputting an indication of the subject source location. In some aspects, the techniques described herein relate to a method wherein the outputting of the indication of the subject source location includes displaying the indication of the subject source location. In some aspects, the techniques described herein relate to a method wherein the outputting of the indication of the subject source location includes providing the indication of the subject source location to an ablation device that coordinates the performing of an ablation on the patient. In some aspects, the techniques described herein relate to a method wherein a cardiogram has multiple leads, wherein each lead has a subject region corresponding to the selected subject region and wherein the applying of the region machine learning model applies a lead region machine learning model for each lead to the subject region of that lead to identify a lead subject base region for that lead and applies a lead source location machine learning model for each lead to the lead subject base region for that lead to identify a lead subject source location for that lead, and wherein the subject source location is derived from the lead subject source locations. In some aspects, the techniques described herein relate to a method wherein the region machine learning model is trained using derived regions of base cardiograms labeled with base regions of the base cardiograms. In some aspects, the techniques described herein relate to a method wherein the source location machine learning model is trained using derived regions of base cardiograms labeled with base source locations associated with the base cardiograms. In some aspects, the techniques described herein relate to a method wherein the region machine learning model and the source location machine learning model are supervised or unsupervised machine learning models. In some aspects, the techniques described herein relate to a method wherein a supervised machine learning model is a classifier or regressor.

In some aspects, the techniques described herein relate to a method performed by one or more computing systems for identifying a source location of an arrhythmia of a patient, the method including: displaying a subject cardiogram of the patient that is collected during an arrhythmia episode of the patient; receiving a selection by a user of a subject region of the subject cardiogram; applying a machine learning model to the subject region to identify a subject source location, the machine learning model being trained based on derived regions of base cardiograms that are derived from base regions of the base cardiograms and source locations associated with the base regions; and outputting an indication of the subject source location. In some aspects, the techniques described herein relate to a method wherein the outputting of the indication of the subject source location includes displaying the indication of the subject source location. In some aspects, the techniques described herein relate to a method wherein the outputting of the indication of the subject source location includes providing the indication of the subject source location to an ablation device that coordinates the performing of an ablation on the patient. In some aspects, the techniques described herein relate to a method wherein the machine learning model is trained using derived regions derived from base cardiograms. In some aspects, the techniques described herein relate to a method wherein a base region of a base cardiogram has a base start time and a base end time within that base cardiogram and wherein each derived region is derived from a base region having a derived start time and a derived end time that are derived from the base start time and the base end time of that base region. In some aspects, the techniques described herein relate to a method wherein the derived regions are labeled with base source locations associated with the base cardiograms. In some aspects, the techniques described herein relate to a method wherein a cardiogram has multiple leads, wherein each lead has a lead subject region corresponding to the selected subject region, wherein the applying of the machine learning model applies a lead machine learning model for each lead to the lead subject region of that lead to identify a lead subject source location for that lead, and wherein the subject source location is derived from the lead subject source locations.

In some aspects, the techniques described herein relate to one or more computing systems for identifying a source location of an arrhythmia of a patient, the one or more computing systems including: one or more computer-readable storage mediums that store computer-executable instructions for controlling the one or more computing systems to: display a subject cardiogram of the patient that is collected during an arrhythmia episode of the patient, the subject cardiogram having one or more leads; receive a selection by a user of a subject region of the subject cardiogram; for each of the one or more leads, identify a lead subject source location based on the subject region of that lead and mappings of derived regions of base cardiograms to base source locations of base cardiograms, each derived region derived from a base region of a base cardiogram and mapped to the base source location of that base region; and output one or more indications of the one or more lead subject source locations; and one or more processors for controlling the one or more computing systems to execute the computer-executable instructions. In some aspects, the techniques described herein relate to one or more computing systems wherein the instructions that output display the one or more indications of the one or more lead subject source locations. In some aspects, the techniques described herein relate to one or more computing systems wherein the one or more indications of the one or more lead subject source locations are displayed on a graphic of a heart. In some aspects, the techniques described herein relate to one or more computing systems wherein the instructions that output provide an indication of a subject source location derived from the one or more lead subject source locations to an ablation device that coordinates performing of an ablation on the patient.

In some aspects, the techniques described herein relate to one or more computer-readable storage mediums that store computer-executable instructions for controlling one or more computing systems to identify a base region of a cardiogram to inform a treatment decision a patient, the computer-executable instructions including instructions that: access a subject region of a subject cardiogram collected from a patient; identify a subject base region of the subject cardiogram based on mappings of derived regions of derived cardiograms to base regions of base cardiograms, each derived region being derived from the base region of a base cardiogram to which the derived region is mapped; and output an indication of the subject base region of the subject cardiogram to inform a treatment for the patient.

In some aspects, the techniques described herein relate to one or more computing systems for identifying a source location of an arrhythmia of a patient, the one or more computing systems including: one or more computer-readable storage mediums that store computer-executable instructions for controlling the one or more computing systems to: display a subject cardiogram of the patient that is collected during an arrhythmia episode of the patient; receive a selection by a user of a subject region of the subject cardiogram; apply a machine learning model to the subject region to identify a subject source location, the machine learning model trained based on mappings of derived regions of derived cardiograms to base regions of base cardiograms, each derived region being derived from the base region of a base cardiogram to which the derived region is mapped, each base region associated with a source location; and output an indication of the subject source location; and one or more processors for controlling the one or more computing systems to execute one or more computer-executable instructions.

In some aspects, the techniques described herein relate to one or more computing systems for training a machine learning model for identifying data associated with a subject region of a subject electromagnetic graph of an electromagnetic signal of an electromagnetic source, the one or more computing systems including: one or more computer-readable storage mediums that store: a plurality of mappings that each map a base region of a base electrogram to base data; and computer-executable instructions for controlling the one or more computing systems to: for each of the plurality of mappings, derive a plurality of derived regions from the base region and the base electrogram of that mapping; and for each of the plurality of the derived regions, generate training data that includes the derived regions labeled with the base data of that mapping; and training a machine learning model using the training data wherein the trained machine learning model, when applied to a subject region of a subject electrogram, outputs subject data.

In some aspects, the techniques described herein relate to one or more computing systems for identifying a source location of an arrhythmia of a patient, the one or more computing systems including: one or more computer-readable storage mediums that store computer-executable instructions for controlling the one or more computing systems to: display a subject cardiogram of the patient that is collected during an arrhythmia episode of the patient; receive a selection by a user of a selected subject region of the subject cardiogram; identify one or more derived subject regions that are derived from the selected subject region; for each of a plurality of the derived subject regions, identify a subject source location based on that derived subject region and mappings of derived regions of base cardiograms to base source locations of the base cardiograms, each derived region derived from a base region of a base cardiogram and mapped to the base source location of that base region; and output indications of the subject source locations; and one or more processors for controlling the one or more computing systems to execute one or more computer-executable instructions. In some aspects, the techniques described herein relate to one or more computing systems wherein the instructions that output display the indications of the subject source locations on a graphic of a heart.

In some aspects, the techniques described herein relate to a method performed by one or more computing systems for identifying a base region of an electromagnetic (EM) waveform, the method including: accessing a subject region of a subject EM waveform collected from an EM source; identifying a subject base region of the subject EM waveform based on mappings of derived regions of base EM waveforms to base regions of base EM waveforms, each derived region being derived from the base region of a base EM waveform to which the derived region is mapped; and outputting an indication of base data relating to the subject base region of the subject EM waveform.

Although the subject matter has been described in language specific to structural features and/or acts, it is to be understood that the subject matter defined in the appended claims is not necessarily limited to the specific features or acts described above. Rather, the specific features and acts described above are disclosed as example forms of implementing the claims.

1. A method performed by one or more computing systems for training a machine learning model (ML) for identifying data associated with a region of a cardiogram, the method comprising:
accessing a plurality of mappings that each map a base region of a base cardiogram to base data;
for each of the plurality of mappings,
deriving a plurality of derived regions from the base region and the base cardiogram of that mapping; and
for each of the plurality of the derived regions, generating training data that includes the derived region labeled with the base data of that mapping; and
training the ML model using the training data wherein the trained ML model, when applied to a subject region of a subject cardiogram, outputs base data as subject data for the subject region
wherein a base region of a base cardiogram has a base start time and a base end time within that base cardiogram and wherein the derived regions that are derived from a base region have derived start times and derived end times that are derived from the base start time and the base end time of that base region and
wherein the derived start times and the derived end times for derived regions that are derived from a base region are derived by adding a positive or negative increment to the base start time of that base region and/or adding a positive or negative increment to the base end time of that base region.

2-3. (canceled)

4. The method of claim 1 further comprising:
receiving a subject region of a subject cardiogram; and
applying the trained ML model to the subject region, which outputs subject data.

5. The method of claim 1 wherein the ML model comprises a convolutional neural network that inputs an image of a derived region and outputs the base data.

6. The method of claim 1 wherein the ML model comprises a portion of autoencoder that generates a latent vector representing an image of the derived region and another ML model that inputs the generated latent vector and outputs the base data.

7. The method of claim 1 wherein the ML model is trained using features derived from the derived region the features being one or more of a time-voltage series specifying voltages and time increments of the derived region, images and time-voltage series of portions of the derived region (e.g., QRS complex), length in seconds of various intervals (e.g., R-R interval, QRS complex, T wave, T-Q interval, and Q-R interval) of the derived region, QRS integral of the derived region, maximum, minimum, mean, and variance of voltages of the derived region, a maximal vector of QRS loop and angle of a vector derived from vectorcardiogram of the derived region, and location of a peak (Q peak) or zero crossing relative to a maximum peak (T peak) in an interval of the derived region.

8. The method of claim 1 wherein the ML model comprises an encoder of a transformer adapted to an image to generate an encoding and a neural network inputs the encoding and other features of a feature vector based on the training data.

9. The method of claim 1 wherein the ML model comprises a neural network that inputs a feature vector representing the derived region.

10. The method of claim 9 wherein the feature vector representing the derived region includes a time-voltage series of the derived region.

11. The method of claim 1 wherein at least some of the mappings have a base time range of the base cardiogram that is selected by a person and have base data that is specified based on treatment of an arrhythmia.

12. (canceled)

13. The method of claim 1 wherein a base cardiogram includes multiple leads and wherein a base region of each lead is mapped to base data and wherein a machine learning model is trained for each lead and further comprising:
receiving lead subject regions of multiple leads of a subject cardiogram;
for each of the multiple leads, applying a trained machine learning model for that lead to the lead subject region of that lead, which outputs lead subject data for that lead; and
determining overall subject data based on analysis of the lead subject data for the leads.

14. The method of claim 1 wherein the base data is a source location of an arrhythmia.

15. The method of claim 1 wherein the base data is a region of a cardiogram.

16. The method of claim 1 wherein the base data is a start time and an end time of a region a cardiogram.

17-60. (canceled)
